(19) United States
(12) Patent Application Publication
(10) Pub. No.: US 2011/0130674 A1
(43) Pub. Date: Jun. 2, 2011

(54) Method and System for Regional Assessment of Lung Physiology

(76) Inventors: Igal Kushnir, Pardes Hana (IL); Meir Botbol, Pardes Hana (IL); Alon Kushnir, Herev Le'et (IL)

(21) Appl. No.: 12/085,453

(22) PCT Filed: Nov. 23, 2006
(86) PCT No.: PCT/IL2006/001352

§ 371 Date: Jul. 16, 2009

Related U.S. Application Data
(60) Provisional application No. 60/739,415, filed on Nov. 25, 2005.

(51) Int. Cl. A61B 5/08

(52) U.S. Cl. 600/529

(57) ABSTRACT

The invention provides a system and method for regional assessment of lung physiology. The system includes a plurality of sound transducers configured to be fixed on a surface of the individual over the thorax. A processor is configured to receive signals generated by the transducers and to determine from the signals a value of a parameter in each of one or more regions of the lungs. The method of the invention includes obtaining signals indicative of pressure waves at locations over the thorax; and determining from the signals a value of a parameter in each of the regions of the lungs.

### FIELD OF THE INVENTION

This invention relates to medical devices and methods, and more particularly to such devices and methods for analyzing body sounds.

### BACKGROUND OF THE INVENTION

Regional assessment of lung physiology has been carried out using radionucleotide perfusion also known as the “VQ scan”. In this technique, radioactive particles are either injected into the subject's blood system or the subject is allowed to inhale suspended radioactive particles. X-ray images of the lungs are obtained and one or both of the lungs in the image is divided into two or more regions. A separate analysis of each lung region is then performed. In most regional lung assessments, each of the two lung images is divided into three parts (top, middle and bottom), and an assessment of lung function or physiology in each region is obtained. Typically, regional assessment involves determining the fraction of the total detected radioactivity detected in each region. The amount of radioactivity detected in each part may be correlated with the lung condition in each part.

Body sounds are routinely used by physicians in the diagnosis of various disorders. A physician may place a stethoscope on a person's chest or back and monitor the patient's breathing in order to detect adventitious (i.e. abnormal or unexpected) lung sounds. The identification and classification of adventitious lung sounds often provides important information about pulmonary abnormalities.

It is also known to fix one or more microphones onto a subject's chest or back and to record lung sounds. U.S. Pat. No. 6,139,505 discloses a system in which a plurality of microphones are placed around a patient's chest. The recordings of the microphones during inhalation and expiration are displayed on a screen, or printed on paper. The recordings are then visually examined by a physician in order to detect a pulmonary disorder in the patent. Kompis et al. (Chest, 120(4), 2001) disclose a system in which M microphones are placed on a patient's chest, and lung sounds are recorded. The recordings generate M linear equations that are solved using a least-squares fit. The solution of the system is to used to determine the location in the lungs of the source of a sound detected in the recordings.

U.S. Pat. No. 6,887,208 to Kushnir et al., provides a system and method for recording and analyzing sounds produced by the respiratory tract. Respiratory tract sounds are recorded at a plurality of locations over an individual's thorax and the recorded sounds are processed to produce an image of the respiratory tract. The processing involves determining from the recorded signals an average acoustic energy, at a plurality of locations over the thorax over a time interval from t₁ to t₂. The term “acoustic energy” at a location is used herein to refer to a parameter indicative of or approximating the product of the pressure and the mass propagation velocity at that location. The image may be used to analyze respiratory tract physiology and to detect pathological conditions. Additionally, a time interval can be divided into a plurality of sub-intervals, and an average acoustic energy determined over the thorax for two or more of the sub-intervals. An image of for each of these sub intervals may then be determined and displayed sequentially on a display monitor. This generates a movie showing dynamic changes occurring in the acoustic energy in the respiratory tract over the time interval.

### SUMMARY OF THE INVENTION

The present invention provides a system and method for regional assessment of lung functioning. In accordance with the invention, microphones are affixed to the body surface at a plurality of locations over the thorax, and signals indicative of lung sounds are recorded. The signals are analyzed in order to produce a value of a predetermined parameter at each of two or more locations on the body surface over the lungs. The two or more locations at which the parameter was determined is clustered into groups, where each group consists of locations on the body surface overlying a particular region of the lungs. The regions may correspond to anatomical regions of the lungs, or may be determined independently of the lung anatomy. For each group of locations, a regional assessment of the underlying lung region is obtained based upon the values of the parameter in the group. The regional assessment may be, for example, the sum of the values of the parameter at the locations in the group, the maximum value, the minimum value or an average value. Alternatively, the regional assessment may be the sum of the values of the parameter at the locations in the group divided by the sum of the values of the parameter in all of the groups. In one embodiment, each lung is divided into three regions (top, middle and bottom), and a regional assessment is obtained as explained above for each of the six regions. In another embodiment, the lungs are divided into regions so that each region has the same number of overlying microphones. The regional assessment may be presented in the form of a table. Alternatively, a diagram showing the contours of the lungs and the lung regions is generated, with the value of the regional assessment of each region appearing in that region of the diagram.

In one embodiment, the plurality of locations is locations at which a microphone was placed. Since the locations where the microphones were placed is known, it is known for each microphone over which lung it is located and where over the lung it is located. The microphones over each lung can be divided into groups. For example, the set of microphones over each lung could be divided into top, middle and bottom groups corresponding to the top, middle or bottom regions of the lungs. A regional assessment of each of the six lung regions can then be obtained.

In another embodiment, values of the parameter are calculated at a plurality of locations including one or more locations at which a microphone was not located. Values of the parameter at locations at which a microphone was not placed can be determined, for example, by interpolation of values calculated at the positions of the microphones. It is preferable to determine, for each location at which a value of the parameter was calculated, whether the location overlies the left lung or the right lung. The invention provides a method for locating the boundary between the locations overlying the left and right lungs, and for locating the top and bottom of the lungs.

In one embodiment of the invention, a breathing cycle is divided into two or more time intervals, and a regional assessment of the lungs, is obtained in accordance with the invention for each time interval.

The system of the invention includes a plurality of N transducers (microphones) configured to be attached to an essentially planar region R of the individual's back or chest over the individual's thorax. The transducers are typically embedded in a matrix that permits to affix them easily on the individual's skin. Such a matrix may typically be in the form of a vest or garment for easily placing over the individual's thorax. As may be appreciated, different matrices may be used for differently sized individuals; for different ages, sexes, etc.

Positions in the region R are indicated by two-dimensional position vectors x=(x¹,x²) in a two-dimensional coordinate system defined in the planar region R. The ith transducer, for i=1 to N, is fixed at a position xᵢ in the region R and generates a signal, denoted herein by P(xᵢ,t), indicative of pressure waves in the body arriving at Xᵢ.

In a preferred embodiment, the parameter calculated at each of the plurality locations is an average acoustical energy. The term “acoustic energy” at a location is used herein to refer to a parameter indicative of or approximating the product of the pressure and the mass propagation velocity at that location. U.S. Pat. No. 6,887,208 to Kushnir et al. discloses a system and method for calculating an average acoustic energy at plurality of locations over the lungs from acoustic signals of lung sounds. As disclosed in that patent, an average acoustic energy, denoted herein by \tilde{P}(x,t₁,t₂), at a plurality of positions x in the region R over a time interval from t₁ to t₂ may be generated from the N signals and used to generate an image of the lungs.

In one embodiment of the invention, an average acoustic energy over a time interval from t₁ to t₂ is obtained at a position of one or more of the microphones using the algebraic expression

$$\tilde{P} ( {x}_{i} , {t}_{1} , {t}_{2} ) = {\int }_{t 1}^{t 2} {P}^{2} ( {x}_{i} , t ) d t \qquad ( 1 )$$

where xᵢ is the position of the microphone.

In a more preferred embodiment, an average acoustic energy \tilde{P}(xᵢ,t₁,t₂) over a time interval from t₁ to t₂ is obtained at a plurality of positions xᵢ of the microphones, for example using Equation (1), and then calculating \tilde{P}(x,t₁,t₂) at other locations x by interpolation of the \tilde{P}(xᵢ,t₁,t₂) using any known interpolation method.

In a most preferred embodiment, the interpolation is performed to obtain an average acoustic energy \tilde{P}(x,t₁,t₂) at a position x=(x¹, x²) in the surface R using the algebraic expression:

$$\tilde{P} ( x , {t}_{1} , {t}_{2} ) = {\sum }_{i = 1}^{N} \tilde{P} ( {x}_{i} , {t}_{1} , {t}_{2} ) g ( x , {x}_{i} , σ ) \qquad ( 2 )$$

where g(x,xᵢ,σ) is a kernel satisfying

$$\begin{matrix}{∇}^{2} g = \frac{\partial g}{\partial σ} & ( 3 ) \\ {\sum }_{i = 1}^{N} g ( x , {x}_{i} , σ ) is approximately equal to 1 & ( 4 )\end{matrix}$$

and where xᵢ=(xᵢ¹,xᵢ²) is the position of the ith microphone and σ is a selectable parameter.

For example, the kernel

$$g ( x , {x}_{i} , σ ) = Exp - ( \frac{{( {x}^{1} - {x}_{i}^{1} \sqrt{σ} )}^{2}}{2 σ} ) \cdot Exp - ( \frac{{( {x}^{2} - {x}_{i}^{2} \sqrt{σ} )}^{2}}{2 σ} ) \qquad ( 5 )$$

may be used.

U.S. Pat. No. 6,887,208 to Kushnir et al. discloses generating an image of the lungs from average acoustic energies calculated over a time interval. In a most preferred embodiment of the invention, an image of the lungs is generated from the calculated average acoustic energies. The image is displayed on a display device with the lungs in the image being divided into the lung regions. The regional assessment of the lung regions is displayed together with the image of the lungs.

Thus, in its first aspect, the invention provides a system for regional assessment in two or more regions of an individual's lungs comprising:
(a) a plurality of N transducers, each transducer configured to be fixed on a surface of the individual over the thorax, the ith transducer being fixed at a location xᵢ and generating a signal P(xᵢ,t) indicative of pressure waves at the location xᵢ; for i=1 to N; and
(b) a processor configured to receive the signals P(xᵢ,t) and determine a value of a parameter in each of the regions in a calculation involving one or more of the signals P(xᵢ,t)

In its second aspect, the invention provides a method for regional assessment in two or more regions of an individual's lungs comprising:
(a) obtaining N signals P(xᵢ,t) indicative of pressure waves at the location xᵢ; for i=1 to N; and
(a) determining a value of a parameter in each of the regions in a calculation involving one or more of the signals P(xᵢ,t)

In its third aspect, the invention provides a computer program product comprising a computer useable medium having computer readable program code embodied therein for regional assessment in two or more regions of an individual's lungs the computer program product comprising:

computer readable program code for causing the computer to determine a value of a parameter in each of the regions in a calculation involving one or more signals P(xᵢ,t) indicative of pressure waves at locations xᵢ; for i=1 to N.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a system for carrying out regional assessment in accordance with one embodiment of the invention;

FIG. 2 shows a flow chart for a method of regional assessment in accordance with one embodiment of the invention;

FIG. 3 shows the locations of microphone placement on the back of a subject for regional assessment in accordance with the invention;

FIG. 4 shows regional assessment of a first subject by the method of the invention (FIG. 4a) and by VQ scan (FIG. 4b);

FIG. 5 shows a method for dividing an image of the lungs into regions; and

FIG. 6 shows regional assessment of a second subject by the method of the invention (FIG. 6a) and by VQ scan (FIG. 6b).

### DETAILED DESCRIPTION OF THE INVENTION

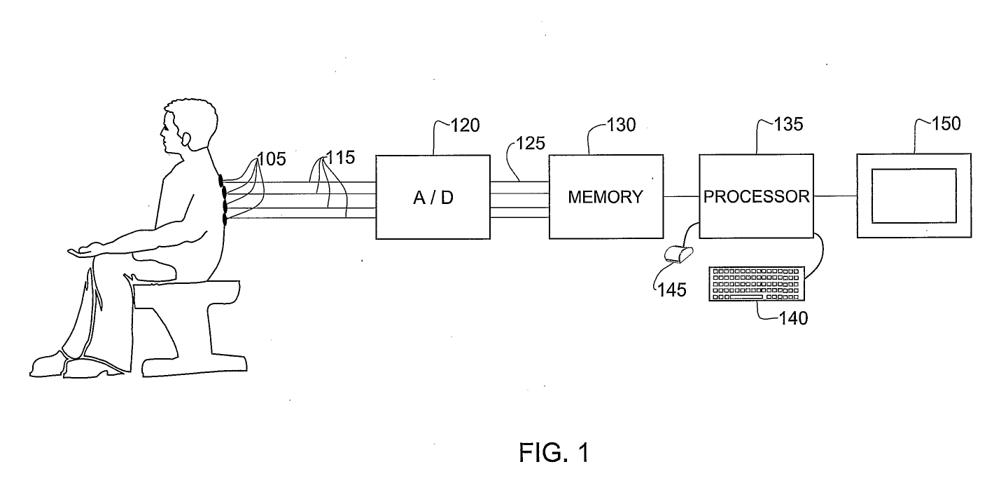

FIG. 1 shows a system generally indicated by 100 for performing regional assessment of the lungs in accordance with one embodiment of the invention. A plurality of N sound transducers 105, of which four are shown, are applied to a planar region of the chest or back skin of individual 110. The transducers 105 may be applied to the subject by any means known in the art, for example using an adhesive, suction, or fastening straps. Each transducer 105 produces an analog voltage signal 115 indicative of pressure waves arriving to the transducer. The analog signals 115 are digitized by a multichannel analog to digital converter 120. The digital data signals P(xᵢ,t) 125, represent the pressure wave at the location xᵢ of the ith transducer (i=1 to N) at time t. The data signals 125 are input to a memory 130. Data input to the memory 130 are accessed by a processor 135 configured to process the data signals 125. The signals 125 may be denoised by filtering components such as components having frequencies outside of the range of lung sounds, for example, vibrations due to movement of the individual. Each signal 125 may also be subject to band pass filtering so that only components in the signal within a range of interest are analyzed.

An input device, such as a computer keyboard 140 or mouse 145, is used to input relevant information relating to the examination such as personal details of the individual 110. The input device 140 may also be used to input values of one or more times t₁ and t₂ that specify times at which the signals P(xᵢ,t) are to be analyzed or that specify one or more time intervals over which no signals P(xᵢ,t) are to be analyzed. The processor 135 calculates the value of a parameter at a plurality of locations over the lungs at the specified times or over the specified time intervals. In a preferred embodiment, the processor 135 is configured to calculate an average acoustic energy \tilde{P}(x,t₁,t₂) over a time interval from t₁ to t₂ at a plurality of locations x in the region R in a calculation involving at least one of the signals P(xᵢ,t).

The locations at which the parameter was calculated are divided into groups, where each group overlies a region of the lungs. The processor 135 is further configured to perform a regional assessment of the lungs. The regional assessment comprises for each of the groups determining the value of one or more regional parameters where each regional parameter is obtained in a calculation involving the parameter values calculated at the location in the region. For example, a regional parameter may be the sum of the parameters in the region, the maximum of the parameter value, the minimum or the average. The regional parameter values may be normalized by dividing the regional parameter by the sum of the regional parameter values.

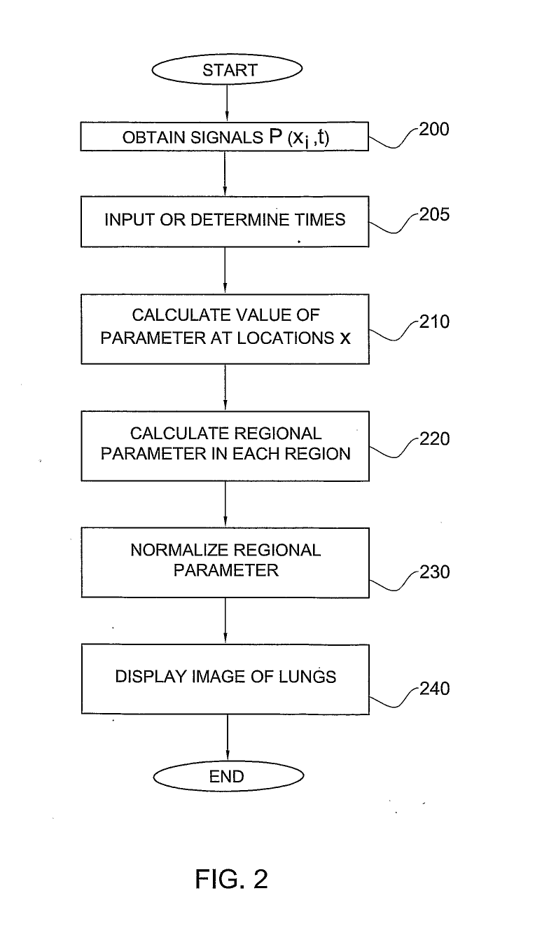

FIG. 2 shows a flow chart diagram for carrying out the method of the invention in accordance with a preferred embodiment. In step 200 the signals P(xᵢ,t) are obtained from N transducers placed at predetermined locations xᵢ for i from 1 to N overlying the lungs. In step 205 values of one or more times are either input to the processor 135 using the input devices 140 or 145, or are determined by the processor. In step 210, a value of a parameter is determined at a plurality of locations x at the one or more input times or over one or more intervals. In step 220 a regional parameter is calculated in each of two or more predetermined lung regions. In step 225, the total of the regional parameters is calculated. In step 230, for each region, the regional parameters are normalized by dividing them by the calculated total to generate the regional assessment of the region. In step 240, an image of the lungs is displayed on the display 150 in which the lungs are divided into the predetermined lung regions, and the normalized or non-normalized regional parameter for each region is displayed in the region in the image. The regional assessment is the total average acoustic energy in the region over the time interval or the total acoustic energy of the region divided by the total acoustic energy of the lungs.

In a most preferred embodiment of the invention, an image of the lungs is generated from the average acoustic energies obtained over a time interval. U.S. Pat. No. 6,887,208 to Kushnir et al. discloses generating an image of the lungs from average acoustic energies calculated at a plurality of locations over the lungs. The image of the lungs is displayed on a display monitor with the lungs in the image being divided into the lung regions.

It will also be understood that the system according to the invention may be a suitably programmed computer. Likewise, the invention contemplates a computer program being readable by a computer for executing the method of the invention. The invention further contemplates a machine-readable memory tangibly embodying a program of instructions executable by the machine for executing the method of the invention.

### EXAMPLES

Two subjects were subjected to regional assessment of lung function by VQ scan and by the method of the invention. The first subject was a 35 year old male having a BMI (body weight to height squared) of 26 who never smoked. The second subject was a 71 year old male having a BMI of 30 who quite smoking five years prior to undergoing regional assessment of lung function. The second subject had a PIY (packs of cigarettes smoked per day times the number of years of to smoking) of 150

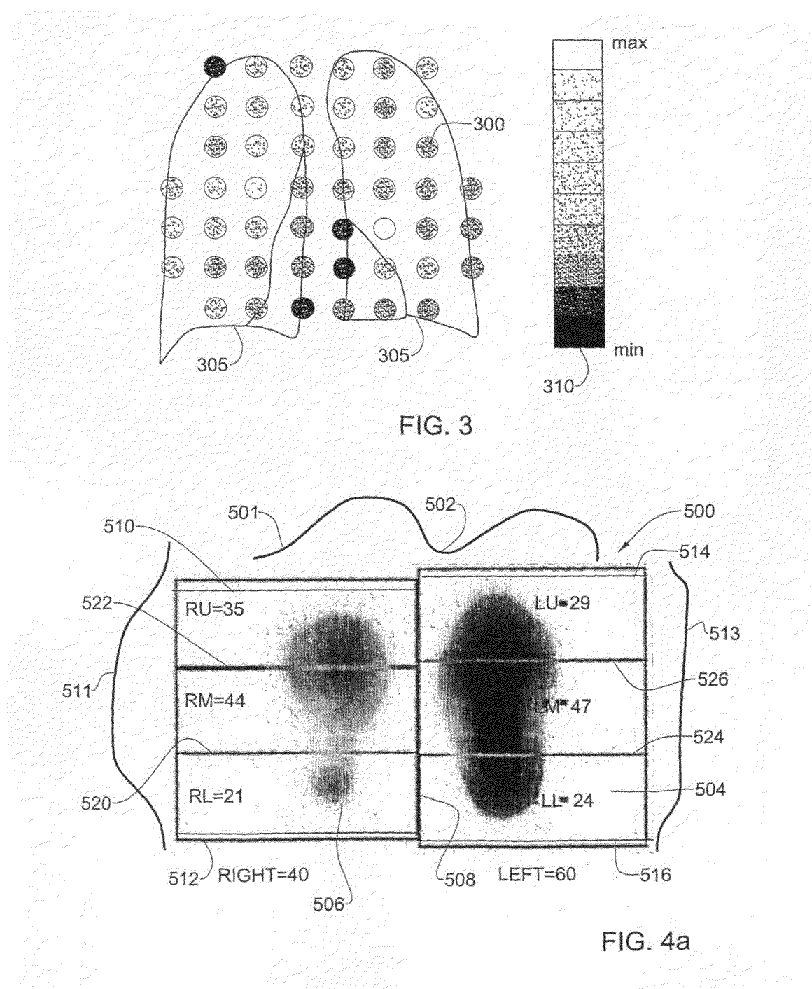

For the regional assessment carried out by the method of the invention, a two-dimensional coordinate system was defined on the subject's back. As shown in FIG. 3a, 48 transducers were placed on the individual's back over the lungs at the locations indicated by the circles 300. The curves 305 show the presumed contours of the lungs. As can be seen, the transducers were arranged in a regular orthogonal lattice with a spacing between the transducers in the horizontal and vertical directions of 5 cm. The signals P(xᵢ,t) were then recorded. Each signal was filtered using a low-pass filter having a cut-off of 150 Hz. The average value of each filtered function P(xᵢ,t) over the respiratory cycle is indicated in FIG. 3a by means of gray level shading of each circle 300 with reference to the gray level scale 310. \tilde{P}(x,t₁,t₂) was obtained using Equations (1) and (2) above with the kernel g of Equation (5) with σ=36 pixels.

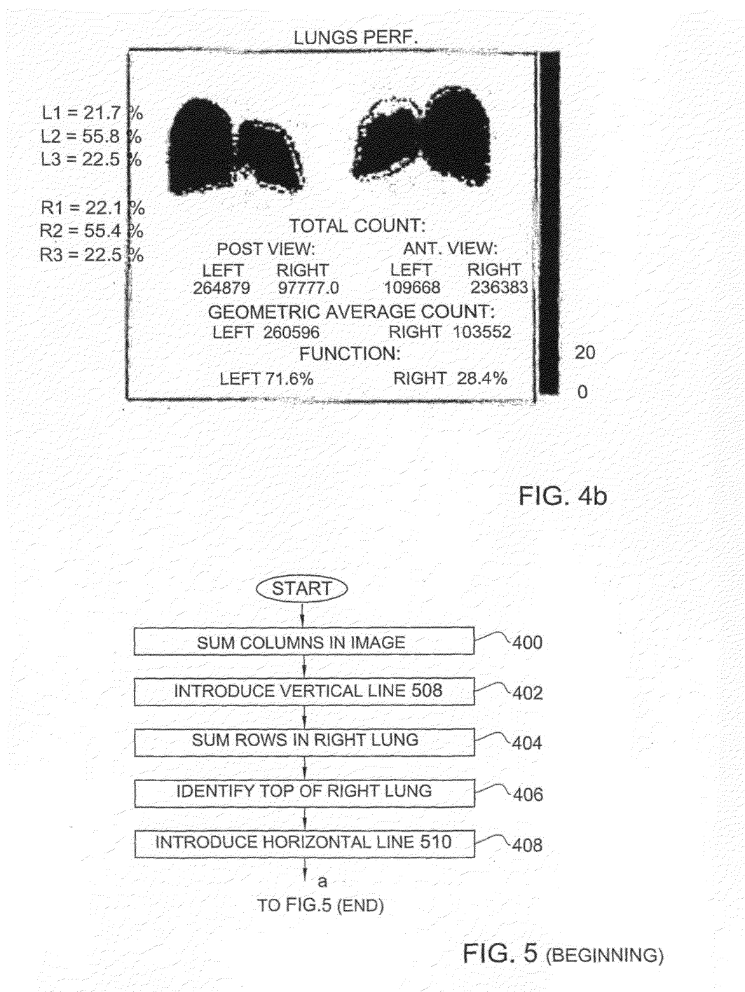

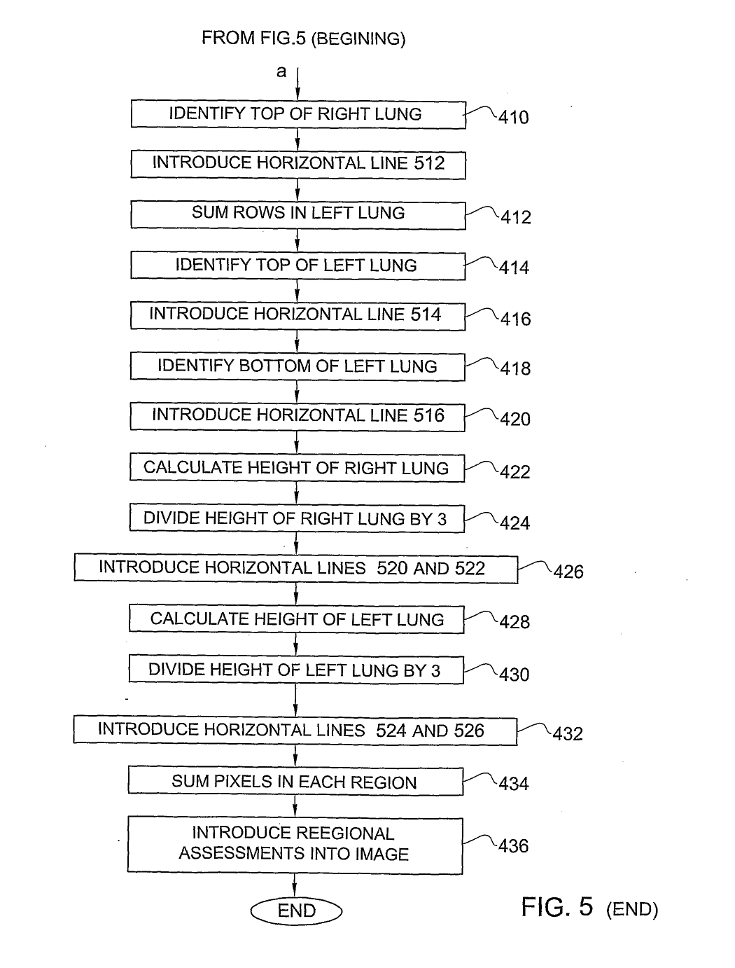

FIG. 4a shows an image 500 of the lungs obtained by the method of U.S. Pat. No. 6,887,208 on the first subject. The image is a two-dimensional array of pixels x(i,j), where x(i,j) is the gray value or other intensity value at the pixel (i,j), where i and j are the column number and row number respectively of the pixel. The image 500 was divided into six regions using the algorithm shown in the flow chart diagram depicted in FIG. 5. In step 400 the intensity values in each column i are summed to yield column sums

$${A}_{i} = {\sum }_{j} x ( i , j ) .$$

The graph 501 of the function Aᵢ is shown in FIG. 4a. The function Aᵢ has a local minimum 502 that identifies the boundary between the left lung 504 and the right lung 506 in the image 500. In step 402 a vertical line 508 is introduced into the image at the boundary between the left and right lungs 504 and 506, respectively.

In step 404 the rows of the image in the right lung are summed to yield row sums

$${B}_{j} = {\sum }_{{i ,}_{{( i , j )}_{in right lung}}} x ( i , j ) .$$

The graph 511 of the function Bⱼ is shown in the image 500 adjacent to the right lung 506. The top of the right lung is identified in step 406 as the highest row j for which Bⱼ exceeds a predetermined threshold value. A horizontal line 510 is then introduced into the image 500 at the top of the right lung in step 408. The bottom of the right lung is identified in step 410 at the lowest row j for which Bⱼ exceeds a predetermined threshold value. A horizontal line 512 is then introduced into the image at the bottom of the right lung in step 410.

In step 412, the rows of the image in the left lung are summed to yield row sums

$${C}_{j} = {\sum }_{{i ,}_{{( i , j )}_{in left lung}}} x ( i , j ) .$$

The graph 513 of the function Cⱼ is shown in the image 500 adjacent to the left lung 504. The top of the left lung is identified in step 414 at the highest row j for which Cⱼ exceeds a predetermined threshold value. A horizontal line 514 is then introduced into the image at the top of the left lung in step 416. The bottom of the left lung is identified in step 418 at the lowest row j for which Cⱼ exceeds a predetermined threshold value. A horizontal line 516 is then introduced into the image at the bottom of the left lung in step 420.

In step 422 the height of the right lung is calculated as the number of pixel rows in the image between the top and bottom of the right lung. In step 424, the height of the right lung is divided by 3 and in step 426, horizontal lines 520 and 522 are introduced into the image 500 so as to divide the right lung in the image into three regions, the right top RT, right middle RM and right bottom RB of equal height.

In step 428, the height of the left lung is calculated as the number of pixel rows in the image between the top and bottom in the left lung. In step 430, the height of the left lung is divided by 3, and in step 432, horizontal lines 524 and 526 are introduced into the image so as to divide the left lung in the image into three regions the left top LT, left middle LM, and left bottom LB of equal height.

Now that the lungs in the image 500 have been divided into the six regions RT, RM, RB, LT, LM, and LB, the intensities of the pixels in each region are summed in step 434. The sum for each region is a value of a regional assessment parameter for the region. In the case that the pixel intensities are calculated as disclosed in U.S. Pat. No. 6,887,208, the regional assessment that is obtained is indicative of the airflow in each region of the lungs.

FIG. 4b shows the regional assessment of the same individual determined by VQ scan. The image was divided into 6 regions and the fraction of radioactivity in each region was calculated, as is known in the art. The regional assessment of each region is shown in the region.

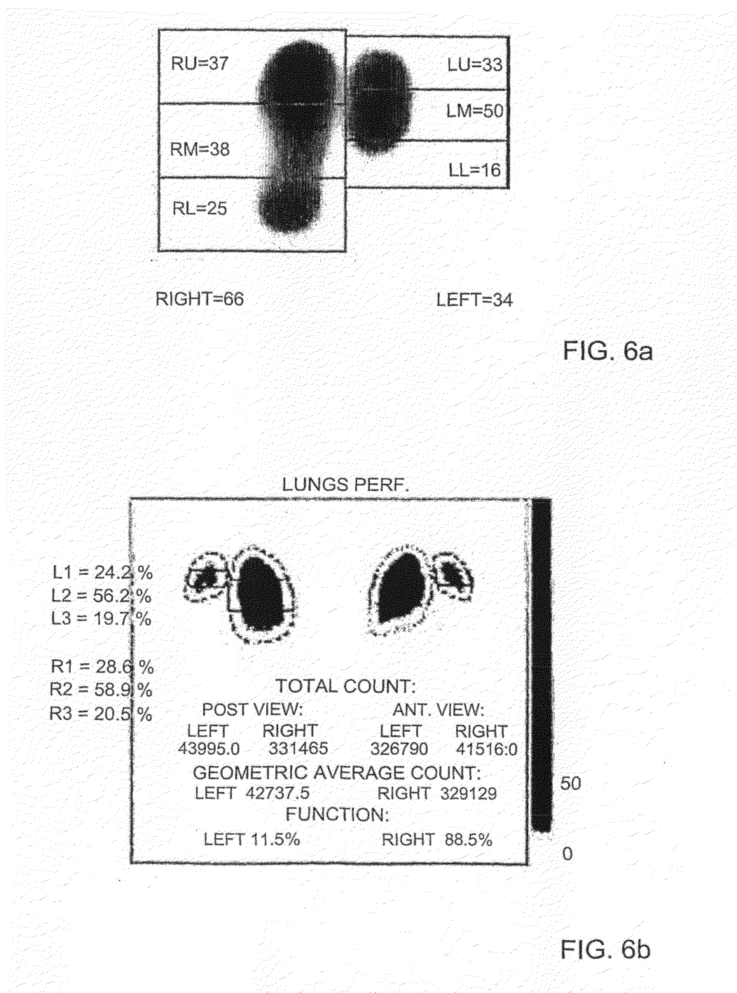

FIG. 6a shows the regional assessment obtained on the second subject by the method of the invention, and FIG. 6b shows the regional assessment obtained on the second subject by VQ scan.

1-27. (canceled)

28. A system for regional assessment in two or more regions of an individual's lungs comprising:
(a) a plurality of N transducers, each transducer configured to be fixed on a surface of the individual over the thorax, the ith transducer being fixed at a location xᵢ and generating a signal P(xᵢ,t) indicative of pressure waves at the location xᵢ; for i=1 to N; and
(b) a processor configured to:
i) receive the signals P(xᵢ,t),
ii) determine a value of a first parameter at a plurality of locations xᵢ of transducers, in a calculation involving one or more of the signals P(xᵢ,t),
iii) determine a value of the first parameter at a plurality of locations x by interpolation of the values determined in step (ii) and
iv) for each of the two or more regions determine a value of a second parameter in a calculation involving the values of the first parameter determined in step (iii) at a plurality of locations x in the region.

29. The system according to claim 28 wherein the first or second parameter is a total average acoustic energy of the region over a time interval from a first time t₁ to a second time t₂.

30. The system according to claim 28 wherein the first or second parameter is a total acoustic energy of the region over a time interval from a first time t₁ to a second time t₂ divided by a total average acoustic energy of the lungs over the same time interval.

31. The system according to claim 30 further comprising a two-dimensional display device.

32. The system according to claim 30 wherein the processor is further configured to display an image of the lungs divided into the regions and the regional assessments of the regions.

33. The system according to claim 32 wherein the image is obtained in a calculation involving the signals P(xᵢ,t).

34. The system according to claim 34 wherein the image is obtained in a calculation involving average acoustic energies \tilde{P}(xᵢ,t₁,t₂) obtained at locations x over the lungs over a time interval from a first time t₁ to a second time t₂.

35. The system according to claim 29 wherein the average acoustic energy \tilde{P} over a time interval from t₁ to t₂ is determined at a location xᵢ of a transducer using the algebraic expression:
$$\tilde{P} ( {x}_{i} , {t}_{1} , {t}_{2} ) = {\int }_{t 1}^{t 2} {P}^{2} ( {x}_{i} , t ) d t .$$

36. The system according to claim 35 wherein an average acoustic energy is determined at least one location x by interpolation of the determined \tilde{P}(xᵢ,t₁,t₂) using the algebraic expression:
$$\tilde{P} ( x , {t}_{1} , {t}_{2} ) = {\sum }_{i = 1}^{N} \tilde{P} ( {x}_{i} , {t}_{1} , {t}_{2} ) g ( x , {x}_{i} , σ ) \qquad ( 2 )$$
where g(x,xᵢ,σ) is a kernel satisfying
$$\begin{matrix}{∇}^{2} g = \frac{\partial g}{\partial σ} & ( 3 ) \\ {\sum }_{i = 1}^{N} g ( x , {x}_{i} , σ ) is approximately equal to 1. & ( 4 )\end{matrix}$$

37. The system according to claim 36 wherein g(x,vᵢσ) is the kernel
$$g ( x , {x}_{1} , σ ) = Exp - ( \frac{{( {x}^{1} - {x}_{i}^{1} \sqrt{σ} )}^{2}}{2 σ} ) \cdot Exp - ( \frac{{( {x}^{2} - {x}_{i}^{2} \sqrt{σ} )}^{2}}{2 σ} ) . \qquad ( 5 )$$

38. The system according to claim 28 wherein the processor is configured to perform the regional assessment of the lungs over a plurality of time intervals.

39. A method for regional assessment in two or more regions of an individual's lungs comprising:
(a) obtaining N signals P(xᵢ,t) indicative of pressure waves at the location xᵢ; for i=1 to N;
(b) determining a value of a first parameter at a plurality of locations xᵢ of transducer, in a calculation involving one or more of the signals P(xᵢ,t);
(c) determining a value of the first parameter at a plurality of locations x by interpolation of the values determined in step (b) and
(d) for each of the two or more regions, determining a value of a second parameter in each of the regions in a calculation involving one or more values of the first parameter determined in step (c) at a plurality of locations x in the region.

40. The method according to claim 39 wherein the first or second parameter is a total average acoustic energy of the region over a time interval from a first time t₁ to a second time t₂.

41. The method according to claim 39 wherein the first or second parameter is a total acoustic energy of the region over a time interval from a first time t₁ to a second time t₂ divided by a total average acoustic energy of the lungs over the same time interval.

42. The method according to claim 39 further comprising a two-dimensional display device.

43. The method according to claim 39 wherein the processor is further configured to display an image of the lungs divided into the regions and the regional assessments of the regions.

44. The method according to claim 43 wherein the image is obtained in a calculation involving the signals P(xᵢ,t).

45. The method according to claim 44 wherein the image is obtained in a calculation involving the average acoustic energies \tilde{P}(xᵢ,t₁,t₂) obtained at locations x over the lungs over a time interval from a first time t₁ to a second time t₂.

46. The method according to claim 45 wherein the average acoustic energy \tilde{P} over a time interval from t1 to t2 is determined at a location xi of a transducer using the algebraic expression:
$$\tilde{P} ( {x}_{i} , t 1 , t 2 ) = {\int }_{t 1}^{t 2} {P}^{2} ( {x}_{i} , t ) d t .$$

47. The method according to claim 45 wherein an average acoustic energy is determined at least one location x by interpolation of the determined \tilde{P}(xᵢ,t₁,t₂) using the algebraic expression:
$$\tilde{P} ( x , {t}_{1} , {t}_{2} ) = {\sum }_{i = 1}^{N} \tilde{P} ( {x}_{i} , {t}_{1} , {t}_{2} ) g ( x , {x}_{i} , σ ) \qquad ( 2 )$$
where g(x,xᵢ,σ) is a kernel satisfying
$$\begin{matrix}{∇}^{2} g = \frac{\partial g}{\partial σ} & ( 3 ) \\ {\sum }_{i = 1}^{N} g ( x , {x}_{i} , σ ) is approximately equal to 1. & ( 4 )\end{matrix}$$

48. The method according to claim 47 wherein g(x,vᵢσ) is the kernel g(x,xᵢ,σ)=
$$Exp - ( \frac{{( {x}^{1} - {x}_{i}^{1} \sqrt{σ} )}^{2}}{2 σ} ) \cdot Exp - ( \frac{{( {x}^{2} - {x}_{i}^{2} \sqrt{σ} )}^{2}}{2 σ} ) . \qquad ( 5 )$$

49. The method according to claim 39 wherein the processor is configured to perform a regional assessment of the lungs over a plurality of time intervals, each regional assessment being determined using an algorithm involving at least one of the signals P(xi,t).

50. A computer program product comprising a computer useable medium having computer readable program code embodied therein for regional assessment in two or more regions of an individual's lungs the computer program product comprising:
computer readable program code for causing the computer to determine a value of a parameter in each of the regions in a calculation involving one or more signals P(xᵢ,t) indicative of pressure waves at locations xᵢ, for i=1 to N.
